(19) United States
(12) Patent Application Publication
(10) Pub. No.: US 2018/0216065 A1
(43) Pub. Date: Aug. 2, 2018

(54) Method for Inducing Differentiation of Myeloid-Derived Suppressor Cells from Cord - Blood CD34 Positive Cells and Proliferating Same, and use of Myeloid-Derived

(71) Applicant: The Catholic University of Korea Industry-Academic Cooperation Foundation, Seoul (KR)

(72) Inventors: Tai Gyu KIM, Seoul (KR); Mi Young PARK, Seoul (KR)

(73) Assignee: The Catholic University of Korea Industry-Academic Cooperation Foundation, Seoul (KR)

(21) Appl. No.: 15/746,773

(22) PCT Filed: Jul. 20, 2016
(86) PCT No.: PCT/KR2016/007912

§ 371 Date: Jan. 22, 2018

(30) Foreign Application Priority Data
Jul. 20, 2015 (KR) 10-2015-0102631

(51) Int. Cl. C12N 5/078; C12N 5/0775

(52) U.S. Cl. CPC C12N 5/0634; C12N 2501/22; C12N 2501/125; C12N 5/0665

(57) ABSTRACT

The present invention relates to a method for inducing differentiation myeloid-derived suppressor cells from cord blood CD34 positive cells and proliferating the same, and a use of the myeloid-derived suppressor cells. More specifically, myeloid-derived suppressor cells are induced to differentiate and proliferated by culturing cord blood CD34 positive cells in the presence of a cytokine cocktail of GM-CSF and SCF, such that myeloid-derived suppressor cells can be mass-produced in vitro, and the myeloid-derived suppressor cells can be used in preventing or treating immunorejection-related diseases such as graft-versus-host disease.

### BACKGROUND

### 1. Field of the Invention

The present invention relates to a method of differentiation induction and proliferation into myeloid-derived suppressor cells from cord blood-derived CD34 positive cells using a cytokine combination, and a use of the myeloid-derived suppressor cells.

### 2. Discussion of Related Art

Graft-versus-host disease (GVHD) can be induced by various factors such as irradiation, the microenvironment of bone marrow, the age and gender of a recipient and a donor, and the source of stem cells. However, most graft-versus-host diseases are caused by the response of transplanted T cells to an incompatible tissue antigen of a recipient (Hill G R et al. Blood, Vol. 90 (8), pp. 3204-13 (1997); Goker H et al., Exp Hematol., Vol. 29 (3), pp. 259-77 (2001)). The subsequent proliferation or activation of other immune cells causes a wide range of damage to tissues of a recipient by cytokine release inducing an inflammatory response (Iwasaki T, Clin Med Res. Vol. 2 (4), pp. 243-52 (2004)). Corticosteroids are generally used as the primary treatment for acute graft-versus-host disease and are more effective when used in combination with immunosuppressants such as cyclosporine and methotrexate. The primary treatment of steroids alleviated lesions of the skin, liver, or gastrointestinal tract and increased the survival rate (1-year prolongation: about 50%) (Ho V T et al., Best Pract Res Clin Haematol., Vol. 21 pp. 223-37 (2008); MacMillan M L et al., Biol Blood Marrow Transplant., Vol. 8 (7), pp. 387-94 (2002)). Patients with graft-versus-host disease who are resistant to steroids receive a secondary treatment such as antithymocyte globulin. However, only 31% of the patients showed an initial improvement of symptoms, and only 10% survived for a long period of time (12 to 60 months). Therefore, a new therapeutic method for improving the survival rate is required. Cord blood-derived regulatory T cells (Treg) or mesenchymal stem cells (MSCs) which were recently proliferated in vitro have been evaluated as a strategy for treating graft-versus-host disease, and the adoptive metastasis of these cells prolonged the survival rate in mice.

Myeloid-derived suppressor cells (MDSCs) have been reported for the first time as inhibiting immune responses in solid cancer as an aggregate of bone marrow-derived immature myeloid cells which suppress functions of immune cells (Murdoch C et al. Nat Rev Cancer., Vol. 8 (8), pp. 618-31 (2008)). Factors that proliferate and activate MDSCs such as stimulating factors such as SCH, VEGF, GM-CSF, G-CSF, and M-CSF; cytokines such as IFN-γ, IL-1b, IL-6, IL-12, and IL-13; calcium binding proteins S100 A8 and S100A9; complement component 3 (C3); cyclooxygenase-2; prostaglandin E2; and the like have been thoroughly studied in tumor models. In healthy subjects, these cells are absent, but they accumulate in peripheral blood, lymphatic tissues, the spleen, cancer tissues, and the like, under pathological conditions such as infection, inflammatory response, cancer, autoimmunity, and the like. Myeloid-derived suppressor cells are defined as CD11b⁺Gr1⁺ cells in mice and as Lin-HLA-DR-CD11b⁺CD33⁺ in humans. These cells are a very heterogeneous (composed of several different types) myeloid cell population, and they are one type of the precursors of hematopoietic stem cells, which develop macrophages, dendritic cells, and granulocytes at various stages of hematopoietic differentiation. In particular, these cells are classified into two groups, monocytic and granulocytic. These two subtypes are distinguished depending on whether CD14 is expressed in humans or Ly6C and Ly6G are expressed in mice.

It was recently reported that adoptive metastasis using MDSCs induced in vitro from mouse embryonic stem cells or hematopoietic stem cells induced immune tolerance in transplantation in mice (Zhou Z et al., Stem Cells., Vol. 28(3), pp. 620-32 (2010); Highfill S L et al. Blood, Vol. 116(25), pp. 5738-47 (2010)). However, even though such a protective effect is reported, the proliferation of MDSCs is even more important for clinical treatment. On the other hand, a combination therapy of MDSCs with other immunoregulatory cells or immunosuppressive drugs has become a highlighted treatment method not only for treating transplant patients, but also for treating patients with allergic reactions and autoimmune diseases. However, factors that induce the accumulation of human-derived MDSCs by induction of immune tolerance in transplantation have not been investigated yet, and methods for mass production of these human-derived MDSCs have not been reported.

Accordingly, the present inventors stably mass-produced cord blood-derived CD34⁺ cells in vitro using GM-CSF and SCF, and completed the present invention by establishing the efficacy of MDSCs in a graft-versus-host disease model using xenogeneic transplantation by transplanting human peripheral blood mononuclear cells (PBMCs) into immunodeficient animals.

### SUMMARY OF THE INVENTION

An object of the present invention is to provide a composition and a method for inducing differentiation and proliferating into myeloid-derived suppressor cells from cord blood-derived CD34⁺ cells.

Another object of the present invention is to provide the myeloid-derived suppressor cells which are differentiated from cord blood-derived CD34⁺ cells and proliferated.

Still another object of the present invention is to provide a pharmaceutical use of the myeloid-derived suppressor cells.

In order to achieve the above objects, the present invention provides a composition for inducing differentiation and proliferating into myeloid-derived suppressor cells (MDSCs) from cord blood-derived CD34⁺ cells, including GM-CSF and SCF.

The present invention also provides a method for inducing differentiation and proliferating into myeloid-derived suppressor cells from cord blood-derived CD34⁺ cells, including culturing cord blood-derived CD34⁺ cells under GM-CSF and SCF to induce differentiation and to proliferate into MDSCs.

The present invention also provides a myeloid-derived suppressor cell, which is differentiated from a cord blood-derived CD34⁺ cell and proliferated, expresses cellular phenotypes of Lin⁻, HLA-DR^{low}, and CD11b⁺CD33⁺, and expresses PDL-1, CCR2, CCR5, CD62L, CXCR4, and ICAM-1 as cell surface markers.

The present invention also provides an immunosuppressive composition, including the myeloid-derived suppressor cell which is monocytic.

According to the present invention, by culturing cord blood-derived CD34⁺ cells under GM-CSF and SCF for a certain period of time, inducing differentiation and proliferating into myeloid-derived suppressor cells from the cord blood-derived CD34⁺ cells, it is possible to mass-produce myeloid-derived suppressor cells in vitro.

The myeloid-derived suppressor cells can be used for preventing or treating a rejection response in organ transplantation or hematopoietic stem cell transplantation, an autoimmune disease, or an allergic disease, which is caused by a hypersensitive immune response.

### BRIEF DESCRIPTION OF THE DRAWINGS

FIG. 1 shows the results of stable amplification of myeloid-derived suppressor cells (MDSCs) under a combination of GM-CSF and SCF from CD34⁺ cells isolated from cord blood.

FIG. 2 shows the results of analyzing whether CD34⁺ cells isolated from cord blood were differentiated into MDSCs through a flow cytometer after culturing the CD34⁺ cells under the combination of GM-CSF and SCF for 6 weeks.

FIG. 3 shows the results of analyzing the differentiation ability of cells having phenotypes of CD11b⁺CD33⁺ and CD11b⁻CD33⁻ after CD34⁺ cells isolated from cord blood were cultured under the combination of GM-CSF and SCF for 3 weeks, and cells having the phenotypes of CD11b⁺CD33⁺ and CD11b⁻CD33⁻ were isolated and cultured under GM-CSF and SCF for 1 week.

FIG. 4 shows the results of subtype analysis of MDSCs induced by culturing CD34⁺ cells isolated from cord blood for 6 weeks.

FIG. 5 shows the analysis results of the cell surface markers of MDSCs which were induced to differentiate from cord blood-derived CD34⁺ cells.

FIGS. 6A and 6B are the results of measuring whether immunosuppressive proteins of MDSCs which were induced to differentiate from cord blood-derived CD34⁺ cells are expressed.

FIGS. 7A to 7D show the results of confirming in vitro immunosuppressive ability of MDSCs which were induced to differentiate from cord blood-derived CD34⁺ cells. FIG. 7A shows suppression ability of MDSCs which were induced to differentiate from cord blood-derived CD34⁺ cells against in vitro allogeneic immune responses, FIG. 7B shows suppression ability of MDSCs which were induced to differentiate from cord blood-derived CD34⁺ cells against antigen-specific T cell responses, FIG. 7C shows the cytokine secretion ability of MDSCs which were induced to differentiate from cord blood-derived CD34⁺, and FIG. 7D is the result of measuring changes in the number of FoxP3-expressing Treg cells by stimulation of MDSCs which were induced to differentiate from cord blood-derived CD34⁺ cells.

FIGS. 8A to 8H and FIGS. 9 to 11 show the results of measuring the efficacy of MDSCs which were induced to differentiate from cord blood-derived CD34⁺ cells against graft-versus-host disease (GVHD) after administering the MDSCs to a xenogeneic mouse model (xenogeneic GVHD).

FIG. 8A shows the results of movement, the degree of back curvature, hair condition, and skin integrity of mice after administering MDSCs which were induced to differentiate from cord blood-derived CD34⁺ cells, FIG. 8B shows changes in mouse weight, FIG. 8C shows the results of scoring the degree of GVHD, FIG. 8D shows changes in the survival rate of the mice, and FIG. 8E to 8H show the results of ELISA analysis of the secretion of cytokines in the mouse serum.

FIG. 9 shows changes in the number of FoxP3-expressing Treg cells.

FIG. 10 shows changes in the intracellular secretion of inflammatory cytokines in mice.

FIG. 11 shows changes in the secretion of anti-inflammatory proteins in the mouse serum.

### DETAILED DESCRIPTION OF EXEMPLARY EMBODIMENTS

Hereinafter, the constitution of the present invention will be described in detail.

The present invention relates to a composition for inducing differentiation and proliferating into myeloid-derived suppressor cells (MDSCs) from cord blood-derived CD34⁺ cells, including GM-CSF and SCE

In addition, the present invention provides a method for inducing differentiation and proliferating into myeloid-derived suppressor cells from cord blood-derived CD34⁺ cells, including culturing cord blood-derived CD34⁺ cells under GM-CSF and SCF to induce differentiation and to proliferate into MDSCs.

The differentiation induction and the proliferation of cord blood-derived CD34⁺ cells into myeloid-derived suppressor cells according to the present invention may mass-produce monocytic myeloid-derived suppressor cells in vitro by culturing the CD34⁺ cells in a cell culture medium including a cytokine combination of GM-CSF and SCF for a certain period of time.

CD34⁺ cells used to induce differentiation into myeloid-derived suppressor cells according to the present invention may be isolated from human cord blood.

The CD34⁺ cells may be isolated by a conventional isolation method, for example, by using a human anti-CD34 antibody.

The myeloid-derived suppressor cells of the present invention may be amplified and differentiated by culturing the CD34⁺ cells in a cell culture medium including GM-CSF and SCF for 2 weeks to 7 weeks, more specifically, for 3 weeks to 6 weeks.

The cell culture medium may be a safe medium for animal cell culture. Examples of the cell culture medium may include Dulbecco's Modified Eagle's Medium (DMEM), Minimal Essential Medium (MEM), Basal Medium Eagle (BME), RPMI1640, F-10, F-12, a Minimal Essential Medium (aMEM), Glasgow's Minimal Essential Medium (GMEM), Iscove's Modified Dulbecco's Medium, and the like, but the present invention is not limited thereto.

The GM-SCF and SCF may be added to a cell culture medium at a concentration ratio of 1:0.8 to 0.3.

Preferably, the GM-CSF may be added to a cell culture medium at a concentration of 50 ng/mL to 200 ng/mL. The SCF may be added to a cell culture medium at a concentration of 10 ng/mL to 100 ng/mL. Within these ranges, the proliferation of CD34⁺ cells may be relatively increased. According to an embodiment, when CD34⁺ cells are cultured in G-CSF/SCF for 3 weeks, the number of the CD34⁺ cells may be multiplied approximately 600-fold, but in GM-CSF/SCF, the number of the CD34⁺ cells may be multiplied 1,000 to 3,000-fold.

Culturing of the CD34⁺ cells for inducing differentiation into myeloid-derived suppressor cells may be maintained for 2 weeks to 7 weeks, more preferably, for 3 weeks to 6 weeks, but the present invention is not limited thereto. According to an embodiment, when cultured for 3 weeks to 6 weeks, differentiation into myeloid-derived suppressor cells having 30% to 95% CD11b⁺CD33⁺ expression may be induced.

The differentiation of the CD34⁺ cells into myeloid-derived suppressor cells may be carried out in a CO₂ incubator under conditions of a 5% to 15% carbon dioxide airflow quantity at 35 to 37° C., but the present invention is not particularly limited thereto.

Under the conditions, differentiation-induced and proliferated myeloid-derived suppressor cells may be proliferated to a cell number of 1,000 to 3,000-fold of an initial number of CD34⁺ cells during culturing.

In the present specification, the term “myeloid-derived suppressor cell (MDSC)” refers to an immature myeloid cell which is present in an immature state because of a granulocyte or the like not completely differentiated in tumors, autoimmune diseases, and infections, and it was reported that the number of MDSCs increases in patients with an acute inflammatory disease, trauma, septicemia, or a parasitic or mycotic infection, as well as in cancer patients. The function of MDSCs is to effectively suppress activated T cells. It is known that the mechanism by which MDSCs regulate T cells is that a nitric oxide synthase, reactive oxygen species (ROS), and arginase which are an enzyme suppress T cell activation by maximizing the metabolism of L-arginine which is an essential amino acid.

The myeloid-derived suppressor cells of the present invention, which were induced to differentiate from the CD34⁺ cells isolated from cord blood, may be monocytic myeloid-derived suppressor cells expressing cellular phenotypes of Lin⁻, HLA-DR^{low}, and CD11b⁺CD33⁺.

The myeloid-derived suppressor cells may express PDL-1, CCR2, CCR5, CD62L, CXCR4, and ICAM-1 as cell surface markers. According to an embodiment of the present invention, when the CD34⁺ cells isolated from cord blood were cultured in GM-CSF and SCF for 6 weeks and the cell surface thereof was stained, 70% HLA-ABC, 30% or less HLA-DR, and at least 90% CD45 were expressed, and compared to MDSCs whose differentiation was induced in a combination of G-CSF/SCF, 10% expression of CD83 and CD80 were observed only in the MDSCs whose differentiation was induced in a GM-CSF/SCF combination according to the present invention. CD86 was expressed at about 40% in the MDSCs of a GM-CSF/SCF combination, which showed an aspect of low expression of co-stimulatory molecules. In addition, CD40 was expressed at 40%, and CD1d, CD3, and B220, which are lymphocyte markers, were expressed at less than 5%. PDL-1 which is known to suppress the proliferation or activation of T cells was expressed at about 30% only in cells cultured in the GM-CSF/SCF combination. CD13 is a transmembrane glycoprotein which is expressed in a myeloid precursor, myeloperoxidase (MPO) is a protein in azurophilic granules of myeloid cells, and both are proteins which are expressed in MDSCs. The expression of CD13 was significantly increased in MDSCs induced by a GM-CSF/SCF combination compared to MDSCs induced by a G-CSF/SCF combination. MPO was expressed at 90% or more in all of the MDSCs induced by two different combinations.

In addition, MDSCs induced by the combination of GM-CSF/SCF increase the expression of an immune suppressor substance selected from the group consisting of arginase 1, indoleamine 2,3-dioxygenase (IDO), and inducible nitric oxide synthase (iNOS), compared to MDSCs induced by a combination of G-CSF/SCF and human peripheral blood-derived dendritic cells.

MDSCs induced by the combination of GM-CSF/SCF significantly suppress the proliferation of allogeneic CD4 T cells and thereby strongly reduce the secretion of IFN-γ by antigen-specific T cell immune responses.

It was observed that MDSCs induced by the combination of GM-CSF/SCF showed a significant increase in the secretion of IL-10 when stimulated with CD40 antibodies, and large amounts of VEFG and TGF-β were secreted without being affected by whether or not stimulated with the CD40 antibodies.

When CD4 T cells are stimulated by MDSCs in vitro, it is known that the number of Treg cells expressing FoxP3 increases, and when CD4 T cells are stimulated by MDSCs induced by a combination of GM-CSF/SCF, FoxP3 expression is confirmed, but IL-17 which is an inflammatory cytokine is not secreted.

In addition, MDSCs induced by the combination of GM-CSF/SCF alleviate the degree of graft-versus-host disease, increases the survival rate, increases the secretions of serum anti-inflammatory cytokines, IL-10, and TGF-0, increases the secretions of anti-inflammatory proteins, CRP, MIP-3β, MMP-9, RANTES (CCL5), and SDF-1a, and suppresses inflammatory responses by reducing the secretions of inflammatory cytokines, IL-17, and IFN-γ, in an animal model for graft-versus-host disease. Moreover, the number of Treg cells expressing FoxP3 is increased.

Therefore, the present invention provides a myeloid-derived suppressor cell, which is differentiated from a cord blood-derived CD34⁺ cell and proliferated, expresses cellular phenotypes of Lin⁻, HLA-DR^{low}, and CD11b⁺CD33⁺, and expresses PDL-1, CCR2, CCR5, CD62L, CXCR4, and ICAM-1 as cell surface markers.

In addition, the present invention provides an immunosuppressive composition, including the myeloid-derived suppressor cell which is monocytic.

The myeloid-derived suppressor cell of the present invention may be used to prevent or treat a rejection response in organ transplantation or hematopoietic stem cell transplantation; an autoimmune disease; or an allergic disease, which is caused by a hypersensitive immune response.

The immunosuppressive composition according to the present invention may further include a pharmaceutically acceptable carrier.

The pharmaceutically acceptable carrier includes a carrier and a vehicle commonly used in the field of medicine, and specifically includes an ion exchange resin, alumina, aluminum stearate, lecithin, a serum protein (e.g., human serum albumin), buffer substances (e.g., various phosphates, glycine, sorbic acid, potassium sorbate, and partial glyceride mixtures of saturated vegetable fatty acids), water, salts or electrolytes (e.g., protamine sulfate, disodium hydrogen phosphate, potassium hydrogen phosphate, sodium chloride, and zinc salts), colloidal silica, magnesium trisilicate, polyvinyl pyrrolidone, cellulose-based substrates, polyethylene glycol, sodium carboxymethyl cellulose, polyarylates, wax, polyethylene glycol, wool grease, or the like, but the present invention is not limited thereto.

In addition to the above components, the composition of the present invention may further include a lubricant, a wetting agent, an emulsifying agent, a suspending agent, a preservative, or the like.

In one aspect, the composition according to the invention may be prepared with an aqueous solution for non-oral administration, and preferably, Hank's solution, Ringer's solution, or a buffer solution such as physically buffered saline may be used. A water-soluble injection suspension may include a substrate capable of increasing the viscosity of a suspension such as sodium carboxymethyl cellulose, sorbitol, or dextran.

The composition of the present invention may be systemically or locally administered and may be formulated into an appropriate dosage form according to known techniques for such administration. For example, for oral administration, the composition may be mixed with an inert diluent or an edible carrier, sealed in a hard or soft gelatin capsule, or formulated as a tablet. In the case of oral administration, the active compound may be mixed with an excipient and used in the form of ingestible tablets, buccal tablets, troches, capsules, elixirs, suspensions, syrups, wafers, or the like.

Various dosage forms for injection, parenteral administration, and the like may be prepared based on techniques known in the art or commonly practiced methods. For administration of a dosage form, intravenous injection, subcutaneous injection, intramuscular injection, peritoneal injection, percutaneous administration, and the like may be used.

The appropriate dosage of the composition of the present invention may be variously prescribed depending on such factors as formulation method, administration method, the age, weight, gender, and morbidity of a patient, food, administration time, administration route, excretion rate, and reaction sensitivity. For example, the composition of the present invention may be administered to adults at a dosage of 0.1 to 1,000 mg/kg, preferably at a dosage of 10 to 100 mg/kg, once or several times daily.

Advantages and features of the present invention and methods of accomplishing the same will become more specific with reference to the exemplary embodiments described in detail below. It should be understood, however, that the present invention is not limited to the exemplary embodiments disclosed below, and may be embodied in various different forms. The exemplary embodiments are only provided so that the disclosure of the present invention will be thorough and complete and the scope of the will be fully conveyed to those of ordinary skill in the art to which the present invention belongs, and the present invention is only defined by the scope of the claims.

### EXAMPLE

### Example 1: Establishment of Stable Amplification of Myeloid-Derived Suppressor Cells by a Cytokine Combination of GM-CSF and SCF in Cord Blood-Derived CD34⁺ Cells

CD34⁺ cells were isolated from cord blood derived from different subjects (humans) by using the MACS method using an antibody against human anti-CD34 (Miltenyi Biotec, Germany) Cord blood-derived mononuclear cells were washed with a MACS buffer. Both an FcR-blocking solution and human CD34 MicroBead (CD34 antibody-bound microbead) were added in an amount of 100 mL per 1×10⁸ cells and were refrigerated for 30 minutes. For separation into CD34-positive cells and CD34-negative cells, after installing a mini-column in a magnetic body, pre-washing was carried out by perfusing 3 mL of a MACS buffer (0.5% BSA, and 2 mM EDTA in PBS at pH 7.2). After the pre-washing, each sample treated with an antibody was resuspended in 1 mL of a MACS buffer to fill the mini-column, CD34-negative cells to which an antibody was not attached were separated by perfusing 3 mL of a MACS buffer three times. After separating the CD34-negative cells, the mini-column was removed from the magnetic body, and CD34-positive cells were separated by perfusing 5 mL of a MACS buffer once. In order to concentrate the separated CD34-positive cells and CD34-negative cells, centrifugation was carried out once using a MACS buffer, and a supernatant liquid was removed.

After collecting CD34 positive cells, the cells were cultured at 1×10⁵ cells per well in a 96-well plate using an IMDM medium with a combination of two kinds of cytokines, that is, GM-CSF (100 ng/mL) and SCF (50 ng/mL), and 3 days later and thereafter, the amplification of CD34⁺ cells was induced in a 48-well plate.

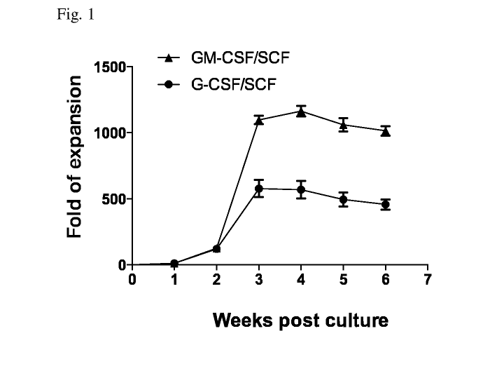

As shown in FIG. 1, with GM-CSF/SCF, there was a 10-fold amplification or more in the first week, a 100-fold amplification or more in the second week, and a 1,000-fold amplification or more in the third week, whereas with G-CSF/SCF, there was a 600-fold amplification in the third week. Therefore, it was found that the combination of GM-CSF (100 ng/mL)/SCF (50 ng/mL) amplified CD34⁺ cells more efficiently.

### Example 2: Analysis of Differentiation-Induced MDSCs after Long-Term Culturing of Cord Blood-Derived CD34⁺ Cells

After separating CD34⁺ cells from cord blood, the CD34⁺ cells were cultured under 37° C. and 5% CO₂ culture conditions in GM-CSF (100 ng/mL)/SCF (50 ng/mL) or G-CSF (100 ng/mL)/SCF (50 ng/mL) for 6 weeks and analyzed to determine whether the CD34⁺ cells were differentiated into myeloid-derived suppressor cells through a flow cytometer.

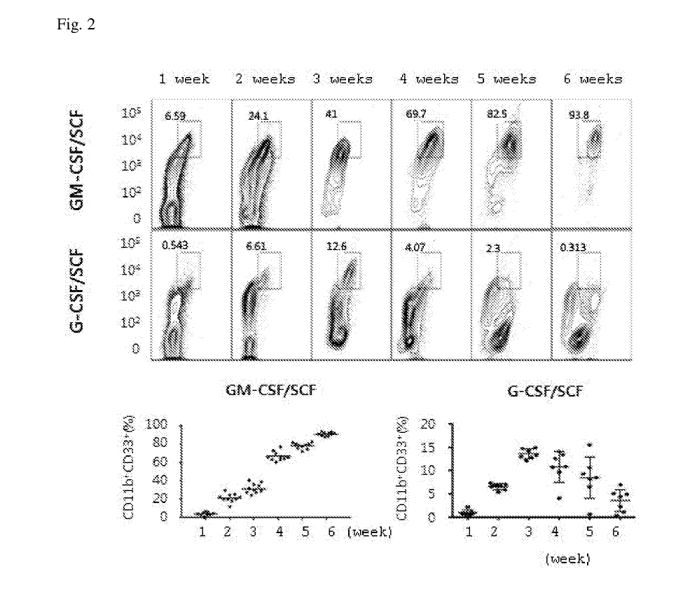

As shown in FIG. 2, as a result of confirming the expression of CD11b⁺CD33⁺ after gating Lin⁻ cells, it was confirmed that with GM-CSF/SCF, there were at least 30% expression of CD11b⁺CD33⁺ after 3 weeks of culturing and about 90% expression of a myeloid-derived suppressor cell group through long-term culturing for 6 weeks. On the other hand, with G-CSF/SCF, there was about 15% expression after 3 weeks, and as suggested by a gradually decreased cell group thereafter, a GM-CSF/SCF cocktail induces the differentiation of MDCSs with high efficiency.

### Example 3: Analysis of Differentiation Ability of CD11b⁺CD33⁺ and CD11b⁻CD33⁻ Cells Induced after Culturing Cord Blood-Derived CD34⁺ Cells for 3 Weeks

After separating CD34⁺ cells from cord blood, the CD34⁺ cells were cultured with GM-CSF (100 ng/mL) and SCF (50 ng/mL) for 3 weeks, and after CD11b⁺CD33⁺ cells and CD11b⁻CD33⁻ cells were separated using FACS Aria, the separated cells were cultured in SCF (50 ng/mL) and GM-CSF (100 ng/mL) for one week and analyzed by a flow cytometer one week thereafter.

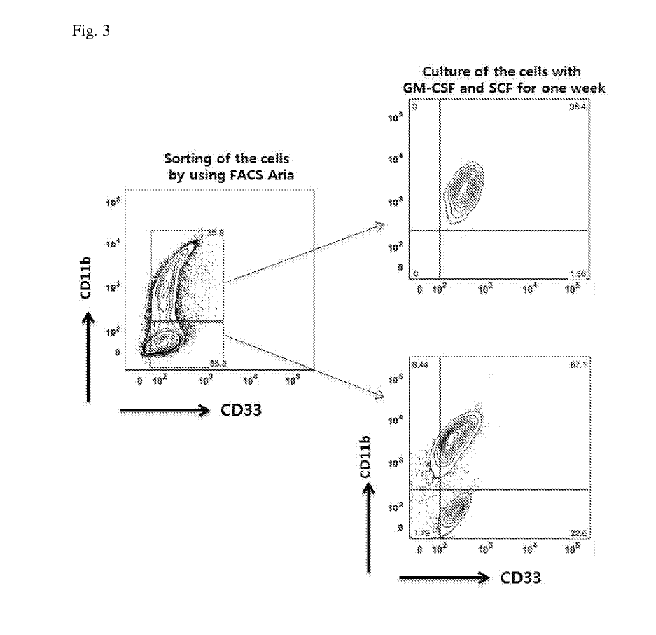

As shown in FIG. 3, as suggested by the fact that the CD11b⁺CD33⁺ cells maintained a 98% CD11b⁺CD33⁺ phenotype and the CD11b⁻CD33⁻ cells showed a 67% CD11b⁺CD33⁺ phenotype, the CD11b⁻CD33⁻ cells were continuously differentiated into CD11b⁺CD33⁺ cells.

### Example 4: Analysis of Differentiation-Induced G-MDSCs and M-MDSCs after Long-Term Culturing of Cord Blood-Derived CD34⁺ Cells

MDSCs are classified into monocytic MDSCs (M-MDSCs) and granular MDSCs (G-MDSCs). Therefore, MDSCs which were induced to differentiate from cord blood-derived CD34⁺ cells were analyzed to determine their subtype.

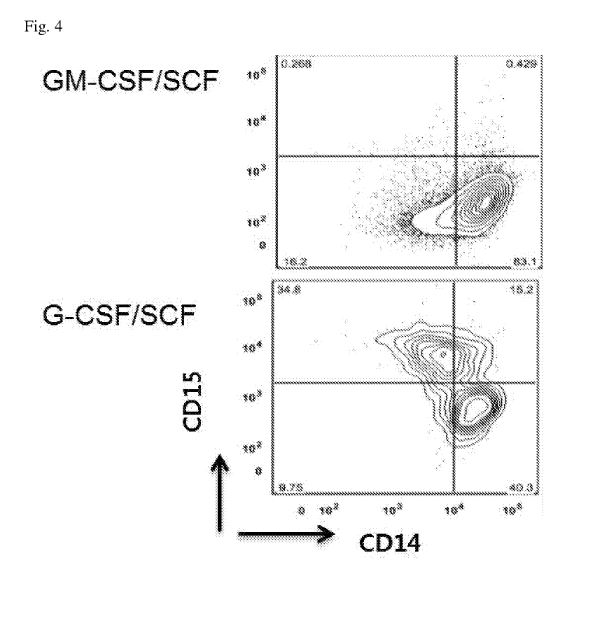

FIG. 4 shows the results of analyzing the expression of CD14 (M-MDSC: CD11b⁺CD33⁺CD14⁺) and CD15 (G-MDSC: CD11b⁺CD33⁺CD15⁺) after culturing CD11b⁺CD33⁺ cells in GM-CSF (100 ng/mL) and SCF (50 ng/mL), or G-CSF (100 ng/mL) and SCF (50 ng/mL) for 6 weeks followed by gating.

As shown in FIG. 4, it was observed that MDSCs which were induced to differentiate from cord blood-derived CD34⁺ cells in GM-CSF (100 ng/mL) and SCF (50 ng/mL) were mostly M-MDSCs as indicated by 83% expression, and MDSCs which were induced to differentiate in G-CSF (100 ng/mL) and SCF (50 ng/mL) were M-MDSCs and G-MDSCs at a ratio of 1:1.

### Example 5: Cell Surface Marker Analysis of MDSCs Induced to Differentiate from Cord Blood-Derived CD34⁺ Cells

After CD34⁺ cells were isolated from cord blood and cultured in GM-CSF (100 ng/mL) and SCF (50 ng/mL) for 6 weeks, cell surface staining was carried out to analyze the cells through a flow cytometer. In this case, cells cultured in G-CSF (100 ng/mL) and SCF (50 ng/mL) were used as a control group.

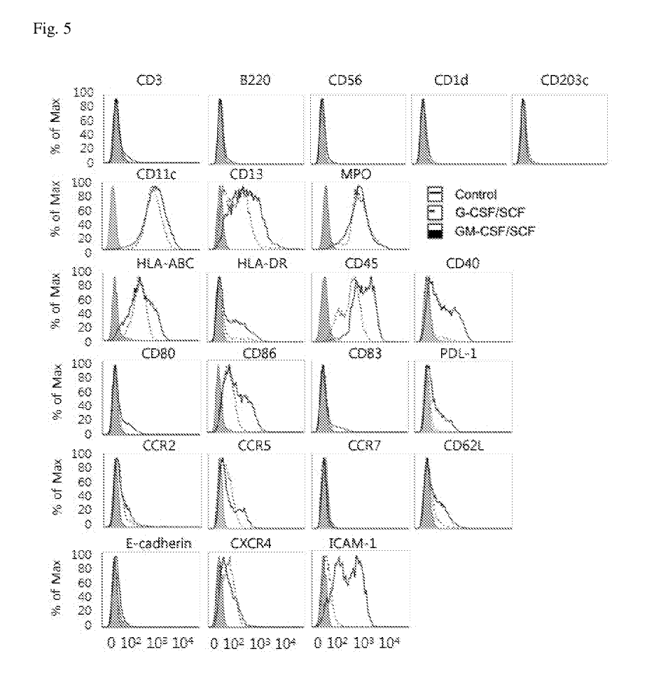

As shown in FIG. 5, 70% HLA-ABC, 30% or less HLA-DR, and at least 90% CD45 were expressed. 10 to 15% CD83 and 20% CD80 were expressed only in cells cultured in GM-CSF/SCF, and 40% CD86 was expressed in cells cultured in GM-CSF/SCF (a significantly higher expression than that of the cells cultured in G-CSF/SCF). Therefore, low expression of co-stimulatory molecules (CD80 and CD86) was observed.

40% CD40, and less than 5% CD1d, CD3, and B220, which are lymphocyte markers, were expressed.

About 30% PDL-1 which is known to suppress the proliferation and activation of T cells was expressed only in cells cultured in GM-CSF (100 ng/mL) and SCF (50 ng/mL).

Next, CD13 is a transmembrane glycoprotein which is expressed in myeloid precursors, myeloperoxidase (MPO) is a protein in azurophilic granules of myeloid cells, and both are proteins which are expressed in myeloid-derived suppressor cells.

As a result of analysis using a flow cytometer, a group of myeloid-derived suppressor cells which were induced by a combination of GM-CSF (100 ng/mL)/SCF (50 ng/mL) showed significantly increased expression of CD13, compared to a group of myeloid-derived suppressor cells which were induced by a combination of G-CSF (100 ng/mL)/SCF (50 ng/mL). It was confirmed that 90% or more MPO was expressed in both of the groups of myeloid-derived suppressor cells which were induced by the two combinations of different cytokines.

In addition, since 88% CD14, 75% CD11c which is a myeloid marker, and 85% CD11b were expressed, it was observed that myeloid cells were highly expressed in cells which were induced to differentiate from cord blood-derived CD34⁺ cells.

### Example 6: Measurement of Expression of Immunosuppressive Proteins in MDSCs Induced to Differentiate from Cord Blood-Derived CD34⁺ Cells

Intracellular signal transduction factors which can determine the suppression ability of MDSCs include arginase 1, iNOS, indoleamine 2,3-dioxygenase (IDO), COX-2, STAT 1, STAT 3, STAT 6, and the like, and such intracellular signal transduction factors were comparatively analyzed in adult PBMC-derived dendritic cells and cord blood-derived MDSCs cultured for 6 weeks.

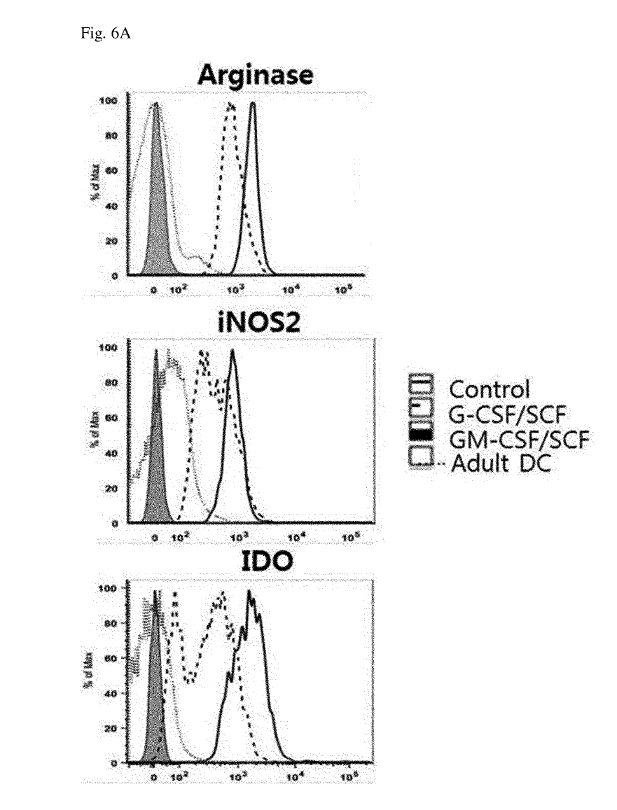

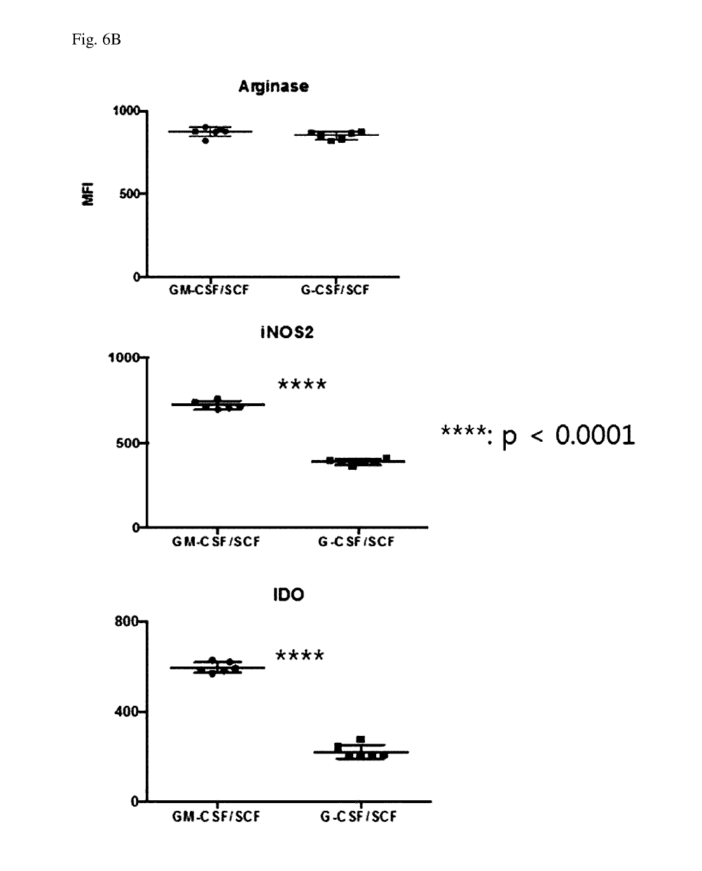

As shown in FIGS. 6A and 6B, as a result of comparing the expression of iNOS2, arginase 1, and IDO in the adult PBMC-derived dendritic cells (adult DCs) and the cord blood-derived MDSCs cultured for 6 weeks, it was observed that the expression of these three molecules was significantly higher in the cord blood-derived MDSCs, and arginase 1 and IDO were slightly increased in adult DCs compared to an unstained state. In addition, the expression of iNOS2 and IDO was significantly higher in GM-CSF/SCF compared to G-CSF/SCF. While arginase 1 was also highly expressed in GM-CSF/SCF compared to G-CSF/SCF, the difference between the two combinations did not show significance.

### Example 7: Evaluation of In Vitro Immunosuppressive Ability of MDSCs Induced to Differentiate from Cord Blood-Derived CD34⁺ Cells

In order to measure the suppression ability of in vitro allogeneic immune responses of cord blood-derived MDSCs, dendritic cells (1×10⁴) and CD4 T cells (1×10⁵, DC:T ratio=1:10), which were isolated from different human individuals, were cultured in a 96-well plate for 4 days. For culturing, the cells were separated into two groups, and cord blood-derived MDSCs (used after 6 weeks of culture in a GM-CSF/SCF combination) were added only to one of the groups. 1 μCi (³H) of thymidine was added to each culture tank, and after 18 hours, measurement was carried out by a liquid scintillation counter.

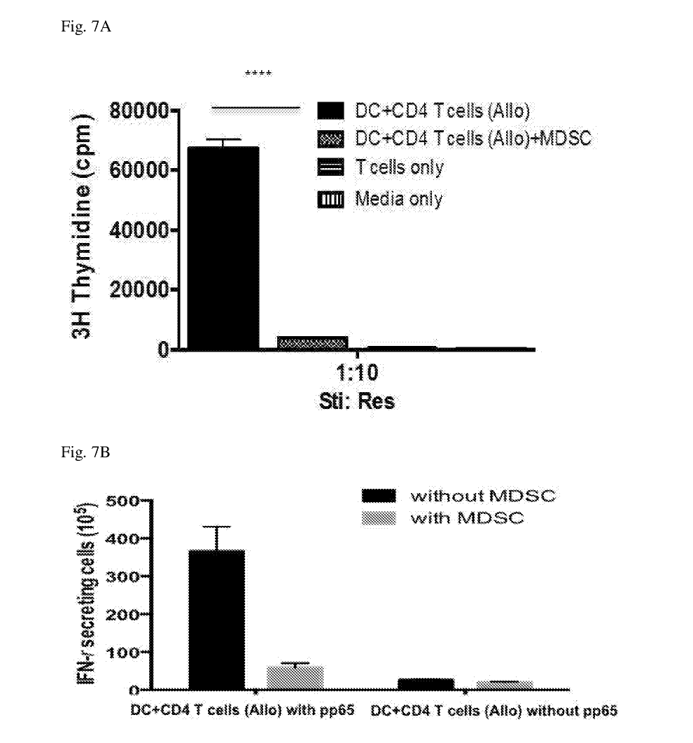

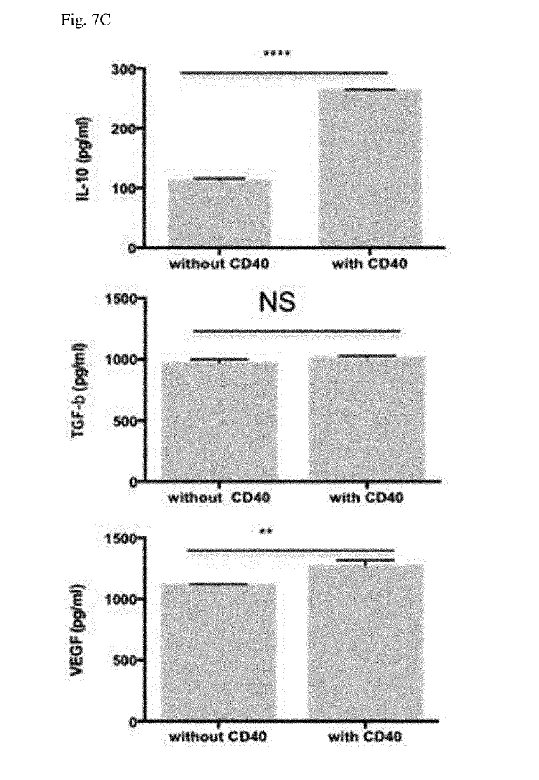

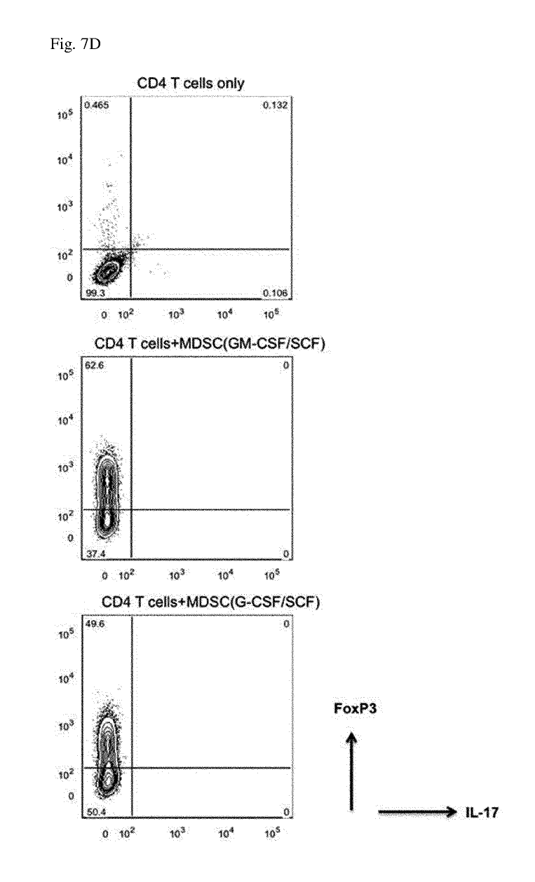

As shown in FIG. 7A, although the dendritic cells efficiently proliferated allogeneic CD4 T cells, the group also including co-cultured cord blood-derived MDSCs suppressed the proliferation of allogeneic CD4 T cells very strongly.

Next, in order to measure whether cord blood-derived MDSCs suppress the antigen-specific T cell response, after dendritic cells with a transfected pp65 antigen or dendritic cells alone (1×10⁴) were reacted with CD4 T cells (1×10⁵, DC:T ratio=1:10) of other donors in a 96-well plate under a condition of the presence or absence of cord blood-derived MDSCs, IFN-γ was measured.

As shown in FIG. 7B, the group cultured with cord blood-derived MDSCs (used after 6 weeks of culturing in a combination of GM-CSF/SCF) very strongly reduced the secretion of IFN-γ due to T cell immune responses specific to the pp65 antigen.

Next, degrees of cytokine secretion of cord blood-derived MDSCs were evaluated.

As shown in FIG. 7C, the secretion of IL-10 was significantly increased when cord blood-derived MDSCs (used after 6 weeks of culturing in a combination of GM-CSF/SCF) were stimulated by the CD40 antibody. Large amounts of VEGF and TGF-β were secreted without being affected by whether or not stimulated with the CD40 antibody.

It has been known that when CD4 T cells are stimulated in vitro with MDSCs, the number of Treg cells expressing FoxP3 increases. Therefore, it was determined whether the number of FoxP3-expressing Treg cells increased due to stimulation with cord blood-derived MDSCs. For this purpose, after culturing 1×10⁵ CD4 T cells with 2×10⁵ MDSCs induced in a combination of GM-CSF/SCF or a combination of G-CSF/SCF at 37° C. and 5% CO₂ for 2 days, cell surfaces were stained using CD3, CD4, and CD25 antibodies, and intracellular staining was carried out using FoxP3 and IL-17A antibodies.

As shown in FIG. 7D, CD4 T cells stimulated with MDSCs induced in a combination of GM-CSF/SCF showed 62% FoxP3 expression, and CD4 T cells stimulated with MDSCs induced in a combination of G-CSF/SCF showed 49% FoxP3 expression. On the other hand, inflammatory cytokine IL-17 was not secreted in both cases.

### Example 8: Evaluation of Efficacy of Cord Blood-Derived MDSCs in Xenogeneic Mouse Model

The efficacy of cord blood-derived MDSCs were determined in a xenogeneic GVHD mouse model. NSG mice which are immunodeficient animals were irradiated at 200 cGY one day before transplantation, and after one day, 1×10⁶ human PBMCs were transplanted in each mouse subject. For the purpose of alleviating graft-versus-host disease (GVHD), 1×10⁶, 2.5×10⁶, and 5×10⁶ cord blood-derived GM-CSF/SCF-induced MDSCs were administered on day 18 and day 24. The weight of the mice was measured once every two days for scoring of graft-versus-host disease, and movements, degrees of back curvature, hair condition, and skin integrity of the mice were observed.

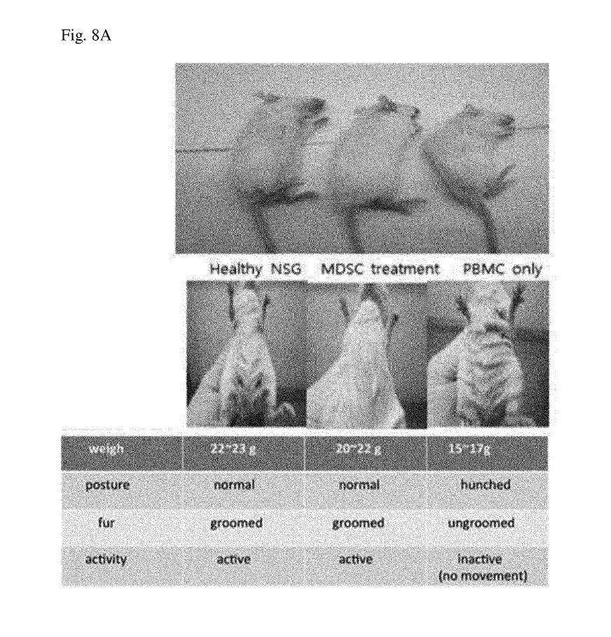

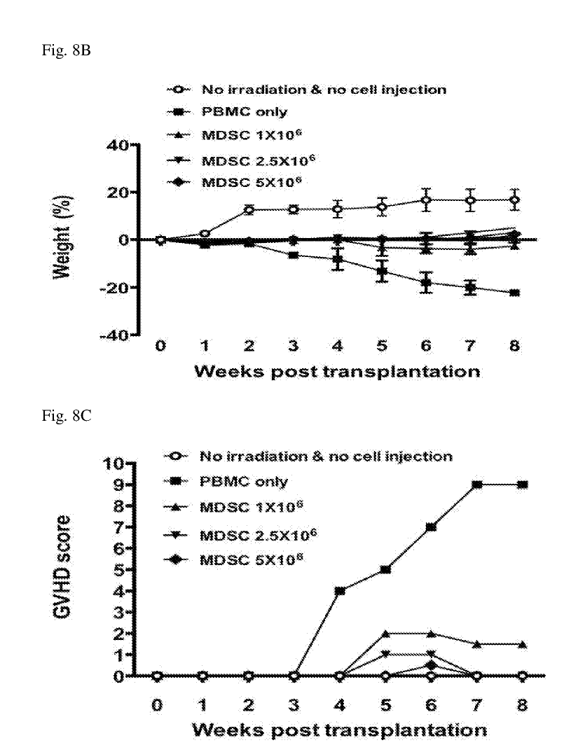

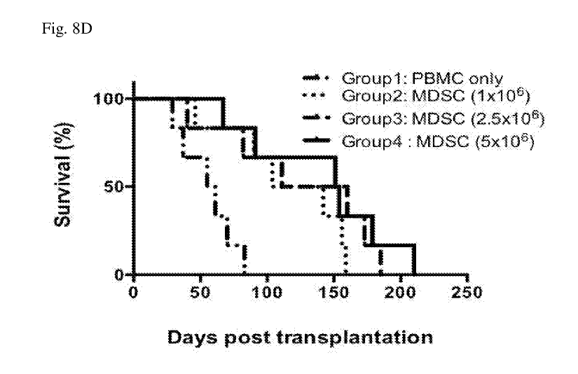

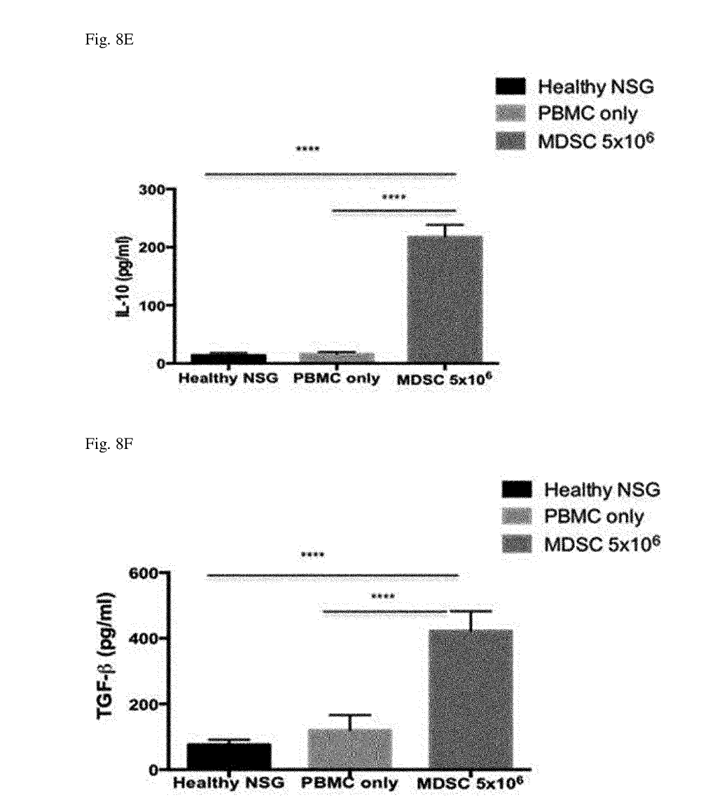

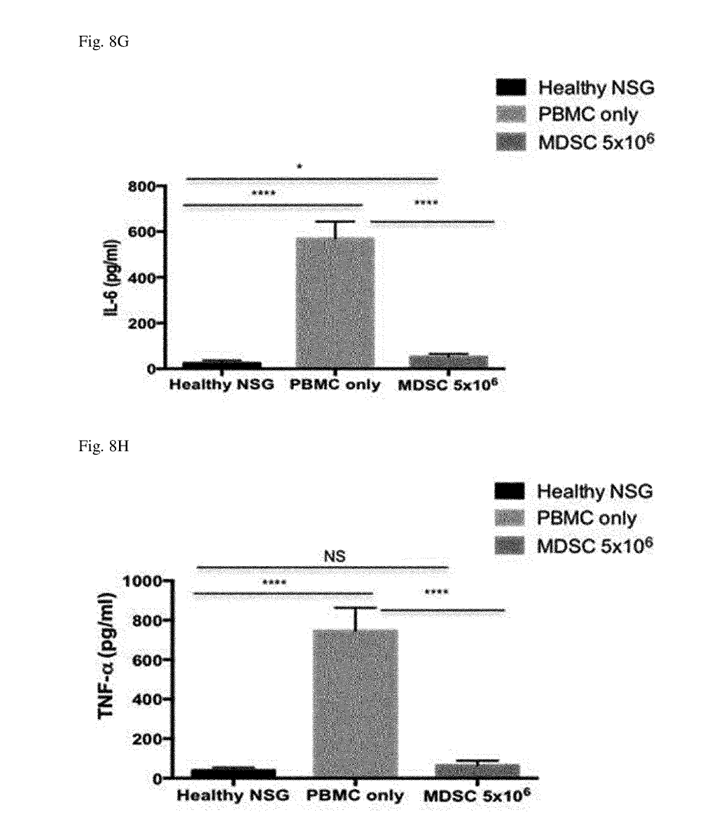

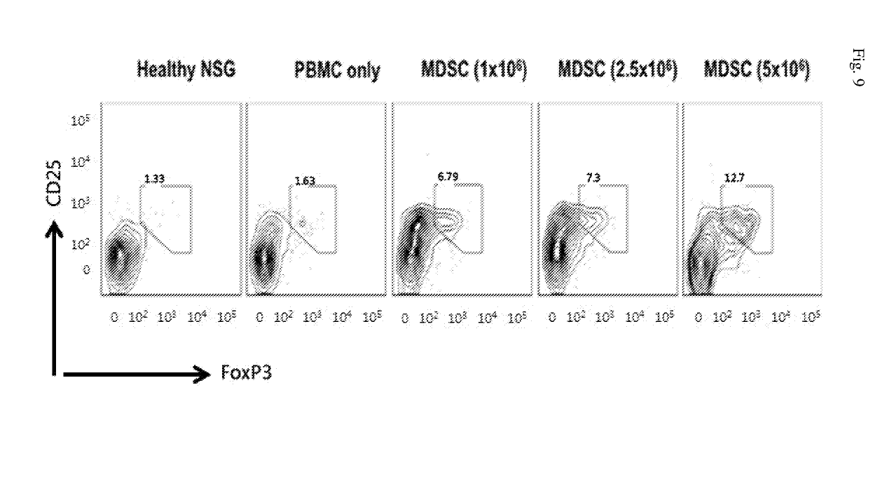

FIG. 8A shows images of mice on day 35 after transplanting human peripheral blood mononuclear cells. Healthy NSG mice (control group) not subjected to radiation and transplantation of human peripheral blood had an average weight of 22 to 23 g, and mice which were administered MDSCs (used after 6 weeks of culturing in a combination of GM-CSF/SCF) had an average weight of 20 to 22 g. On the other hand, mice (PBMCs only) which were administered only human peripheral blood mononuclear cells had an average weight of 15 to 17 g, their back was very curved, and there was no movement at all.

Next, it was determined whether the weight was decreased in the xenogeneic mouse model after administering cord blood-derived MDSCs. The weight was measured at 2-day intervals to show a weight decrease on a graph.

As shown in FIG. 8B, the group administered only PBMCs showed a gradual decrease in weight, and 6 weeks later and thereafter, a weight decrease of about −20% was observed, while the group treated with MDSCs (used after 6 weeks of culturing in a combination of GM-CSF/SCF) showed alleviated weight loss.

Next, the degree of graft-versus-host disease was scored 60 days after PBMC transplantation. As shown in FIG. 8C, the group treated with only PBMCs was assigned 9 points because not only the weight was decreased but also the back was curved 30 degrees or more, hair was generally removed, and there was nearly no movement. On the other hand, the groups treated with MDSCs (used after 6 weeks of culturing in a combination of GM-CSF/SCF) had a lower score as the number of cells increased, and in particular, the group treated with 5×10⁶ MDSCs was assigned 0.5 points. Therefore, it was possible to observe that MDSCs alleviated the degree of GVHD.

Next, a survival rate was measured in the xenogeneic GVHD mouse model after administering cord blood-derived MDSCs.

As shown in FIG. 8D, compared to the group administered only PBMCs, the groups treated with MDSCs (used after 6 weeks of culturing in a combination of GM-CSF/SCF) showed significantly increased survival rates. However, there was no significance in survival rates depending on the number of cells.

MDSCs are known to secret IL-10 which is an anti-inflammatory and immunosuppressive cytokine, TNF-α, IL-1b, and IL-6, which are pro-inflammatory cytokines, and proteins such as VEGF. Therefore, the mouse serum was isolated 35 days after PBMC transplantation to measure anti-inflammatory cytokines by ELISA.

As shown in FIGS. 8E to 8H, compared to the group administered only PBMCs, significant increases in the amounts of IL-10 and TGF-β which are anti-inflammatory cytokines were observed in the group treated with MDSCs (used after 6 weeks of culturing in a combination of GM-CSF/SCF). On the other hand, the amounts of IL-6 and TNF-α which are pro-inflammatory cytokines were significantly increased in the group administered only PBMCs.

It has been known that when CD4 T cells are stimulated in vitro with MDSCs, the number of Treg cells expressing FoxP3 increases. After culturing 1×10⁵ CD4 T cells with 2×10⁵ MDSCs induced in a combination of GM-CSF/SCF or a combination of G-CSF/SCF at 37° C. and 5% CO₂ for 2 days, cell surfaces were stained using CD3, CD4, and CD25 antibodies, and intracellular staining was carried out using FoxP3 and IL-17A antibodies.

As shown in FIG. 9, the number of Treg cells expressing FoxP3 increased relative to the number of CD4 cells stimulated with MDSCs induced in GM-CSF/SCF.

Next, the secretion of intracellular inflammatory cytokines was determined in the xenogeneic GVHD mouse model after administering cord blood-derived MDSCs. For this purpose, cells were isolated from the spleen of the mice 35 days after PBMC transplantation, cell surfaces were stained using CD3 and CD4 antibodies, and intracellular staining was carried out using IL-17, IL-4, and IFN-γ antibodies.

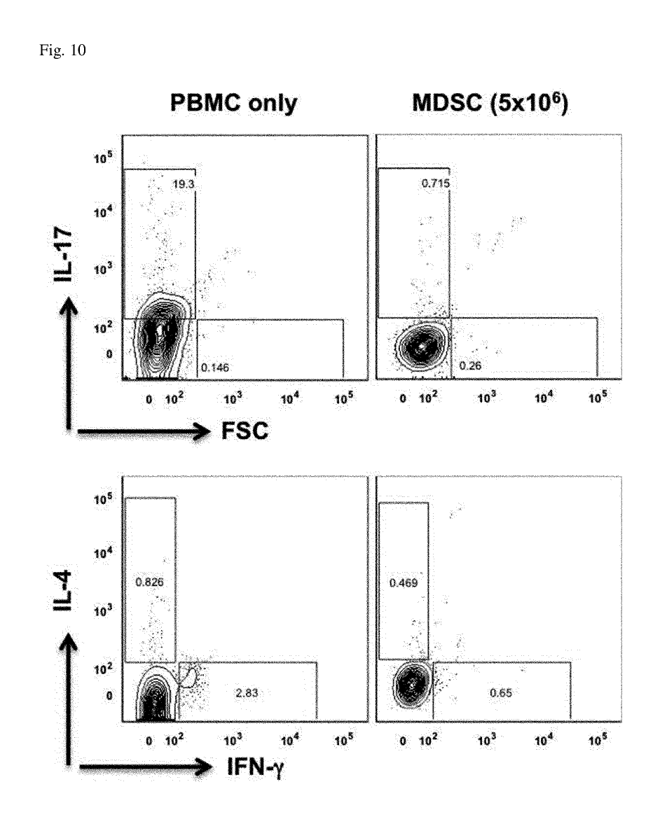

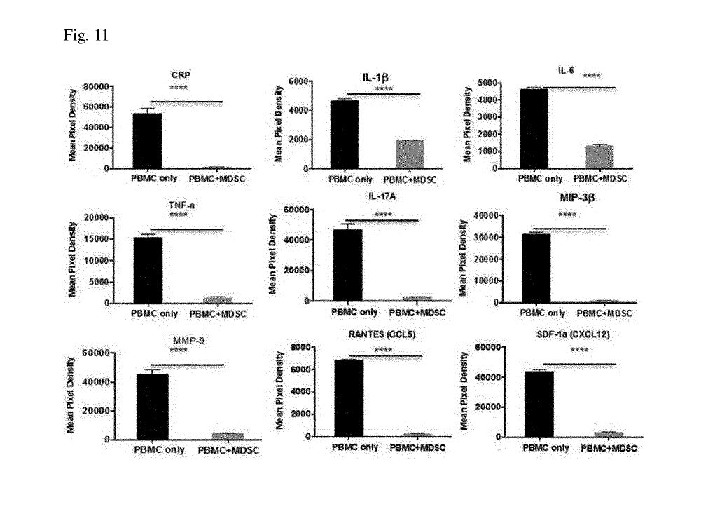

As shown in FIG. 10, the expression of IL-17 and IFN-γ was significantly increased in the group administered only PBMCs. Therefore, it was confirmed that inflammatory responses were suppressed when MDSCs (used after 6 weeks of culturing in GM-CSF/SCF) were administered.

Finally, it was determined whether anti-inflammatory proteins in the serum of the xenogeneic mouse model were secreted after administering cord blood-derived MDSCs. For this purpose, the serum of the mice was isolated 35 days after PBMC transplantation and measured using a cytokine array kit (a kit capable of simultaneously measuring differences in the level of secreted cytokines between samples).

As shown in FIG. 11, inflammatory cytokines and proteins were significantly secreted in the serum of the group administered only PBMCs. On the other hand, it was confirmed that the amounts of inflammatory cytokines and proteins were decreased in the group treated with MDSCs (used after 6 weeks of culturing in a combination of GM-CSF/SCF).

The present invention can be used to prevent or treat a rejection response in organ transplantation or hematopoietic stem cell transplantation, an autoimmune disease, or an allergic disease, which is caused by a hypersensitive immune response.

What is claimed is:

1. A composition for inducing differentiation and proliferating into myeloid-derived suppressor cells (MDSCs) from cord blood-derived CD34⁺ cells, comprising GM-CSF and SCF.

2. The composition of claim 1, wherein the CD34⁺ cells are isolated from human cord blood.

3. The composition of claim 1, wherein the myeloid-derived suppressor cells express cellular phenotypes of Lin⁻, HLA-DR^{low}, and CD11b⁺CD33⁺.

4. The composition of claim 1, wherein the myeloid-derived suppressor cells comprise the expression of PDL-1, CCR2, CCR5, CD62L, CXCR4, and ICAM-1, as cell surface markers.

5. A method for inducing differentiation and proliferating into myeloid-derived suppressor cells (MDSCs) from cord blood-derived CD34⁺ cells, comprising
culturing cord blood-derived CD34⁺ cells under GM-CSF and SCF to induce differentiation and to proliferate into MDSCs.

6. The method of claim 5, wherein the CD34⁺ cells are isolated from human cord blood.

7. The method of claim 5, wherein the GM-CSF and the SCF are added to a cell culture medium at a concentration ratio of 1:0.8 to 0.3.

8. The method of claim 5, wherein the GM-CSF is added to a cell culture medium at a concentration in a range of 50 ng/mL to 200 ng/mL.

9. The method of claim 5, wherein the SCF is added to a cell culture medium at a concentration in a range of 10 ng/mL to 100 ng/mL.

10. The method of claim 5, wherein the differentiation and proliferation of the myeloid-derived suppressor cells are induced by culturing the CD34⁺ cells under the GM-CSF and the SCF for 2 weeks to 7 weeks.

11. The method of claim 5, wherein the myeloid-derived suppressor cells are proliferated to a cell number 1,000 to 3,000-fold of an initial cell number of the CD34⁺ during culturing.

12. The method of claim 5, wherein the myeloid-derived suppressor cells express cellular phenotypes of Lin⁻, HLA-DR^{low}, and CD11b⁺CD33⁺.

13. The method of claim 5, wherein the myeloid-derived suppressor cells comprise the expression of PDL-1, CCR2, CCR5, CD62L, CXCR4, and ICAM-1, as cell surface markers.

14. A myeloid-derived suppressor cell, wherein the myeloid-derived suppressor cell is differentiated from a cord blood-derived CD34⁺ cell and proliferated, and expresses cellular phenotypes of Lin⁻, HLA-DR^{low}, and CD11b⁺CD33⁺, and includes the expression of PDL-1, CCR2, CCR5, CD62L, CXCR4, and ICAM-1, as cell surface markers.

15. The myeloid-derived suppressor cell of claim 14, wherein the myeloid-derived suppressor cell increases the expression of an immune suppressor substance selected from the group consisting of arginase 1, indoleamine 2,3-dioxygenase (IDO), and inducible nitric oxide synthase (iNOS), compared to a human peripheral blood-derived dendritic cell and a myeloid-derived suppressor cell induced by a combination of G-CSF/SCF.

16. An immunosuppressive composition comprising the myeloid-derived suppressor cell of claim 14, wherein the myeloid-derived suppressor cell is monocytic.

17. The immunosuppressive composition of claim 16, wherein the immunosuppressive composition is used for preventing or treating a rejection response in organ transplantation or hematopoietic stem cell transplantation, an autoimmune disease, or an allergic disease, which is caused by a hypersensitive immune response.

18. The immunosuppressive composition of claim 16, wherein the immunosuppressive composition is used for alleviating graft-versus-host disease (GVHD).
